(19) United States
(12) Patent Application Publication
(10) Pub. No.: US 2021/0308180 A1
(43) Pub. Date: Oct. 7, 2021

(54) COMPOSITION FOR PREVENTING OR TREATING COVID-19, INCLUDING GOLD NANOPARTICLE AS ACTIVE INGREDIENT

(71) Applicant: Chur CHIN, Daegu (KR)

(72) Inventor: Chur CHIN, Daegu (KR)

(21) Appl. No.: 16/929,730

(22) Filed: Jul. 15, 2020

Related U.S. Application Data
(63) Continuation-in-part of application No. PCT/KR2020/004639, filed on Apr. 6, 2020.

(51) Int. Cl. A61K 33/242; A61K 9/51; A61K 9/00; A61K 31/155; A61P 31/14

(52) U.S. Cl. CPC A61K 33/242; A61K 9/51; A61P 31/14; A61K 31/155; A61K 9/0043

(57) ABSTRACT

The present invention relates to a pharmaceutical composition for treating or preventing coronavirus infection (COVID-19), containing a gold nanoparticle. In the present invention, the gold nanoparticles hinder the morphological change of a coronavirus protein to block the entry of the coronavirus protein into cells, resulting in prevention or treatment of the coronavirus infection (COVID-19).

### TECHNICAL FIELD

The present invention relates to a composition for preventing or treating coronavirus infection, comprising a gold nanoparticle as an active ingredient.

### BACKGROUND OF THE INVENTION

Coronavirus disease (COVID-19) is an infectious disease caused by a newly discovered coronavirus. Most people infected with the COVID-19 virus will experience mild to moderate respiratory illness. Coronavirus infection mainly results in a respiratory infection that has spread worldwide since it recently occurred in Wuhan, China in December 2019.

As shown in FIG. 1, coronavirus includes a spike protein, and a spike glycoprotein of coronavirus-19 mediates viral entry and membrane fusion to host cells, and thus has become a main target for vaccines and therapeutic products (Artificial Cells, Blood Substitutes, and Biotechnology, vol 40. pp 75-96). However, so far, it was confirmed that most monoclonal antibodies (mAbs) do not neutralize synonymous mutations.

The spike glycoprotein of the coronavirus has a multifunctional molecular structure mediating coronavirus entry into host cells. As shown in FIG. 2, first, the spike glycoprotein binds to a receptor on the host cell surface via an 51 subunit. Two domains of 51 in other coronaviruses are known to induce viral attachment by recognition of various host receptors. Such a spike protein is present in the form of two structurally distinct pre-fusion and post-fusion morphologies.

The present invention is provided to solve the above-described problems, and the inventors demonstrated that a gold nanoparticle hinders the morphological change of corona virus, and is capable of blocking entry into a cell.

However, technical problems to be solved in the present invention are not limited to the above-described problems, and other problems which are not described herein will be fully understood by those of ordinary skill in the art from the following descriptions.

### SUMMARY OF THE INVENTION

The present invention provides a pharmaceutical composition for treating or preventing COVID-19, comprising a gold nanoparticle.

In addition, the present invention provides a method of treating or preventing coronavirus COVID-19, comprising administering a composition containing a gold nanoparticle to a subject. Furthermore, the present invention provides a use of a composition including a gold nanoparticle for treating or preventing COVID-19.

In one embodiment of the present invention, the gold nanoparticle hinders the morphological change of a coronavirus protein to block the entry of a coronavirus protein into a cell.

In another embodiment of the present invention, the gold nanoparticle is included in the form of a negatively-charged gold colloidal solution.

In still another embodiment of the present invention, the composition further includes a S1 proteolysis inhibitor.

In yet another embodiment of the present invention, the composition is a composition for a nasal spray.

In yet another embodiment of the present invention, the gold nanoparticle has a diameter of 0.1 to 100 nm.

A composition according to the present invention contains a gold nanoparticle as an active ingredient, and in the present invention, an effect of treating and preventing COVID-19 according to the treatment of the gold nanoparticle was confirmed. The gold nanoparticle hinders the morphological change of a coronavirus protein, so that the entry of the coronavirus protein into a cell can be blocked, resulting in the prevention or treatment of COVID-19.

### BRIEF DESCRIPTION OF DRAWINGS

FIG. 1 schematically shows the morphology of coronavirus.

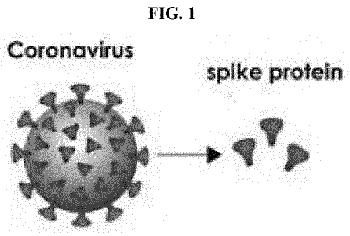

FIG. 2 shows structures before and after fusion due to entry of the coronavirus into a viral host cell.

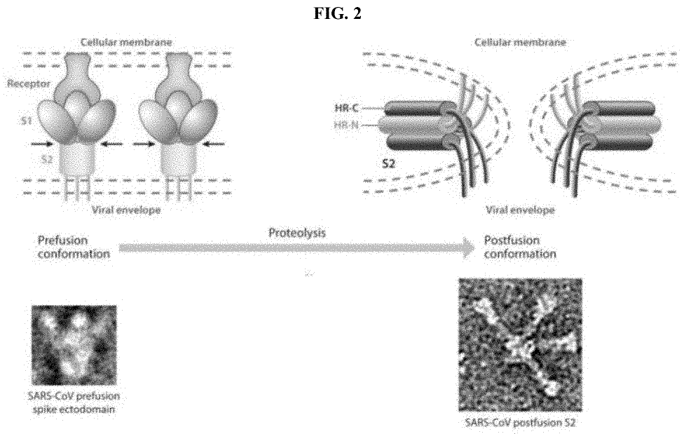

FIG. 3 shows a gold nanoparticle applied to the coronavirus.

FIG. 4 shows that a gold nanoparticle is added to disulfide bonding between spike proteins of the coronavirus.

FIG. 5 schematically shows a process of treating a coronavirus disease using a composition of the present invention binding to the virus.

FIG. 6 shows the morphology of an S1 proteolysis inhibitor-binding gold nanoparticle.

### DETAILED DESCRIPTION OF THE INVENTION

The present invention provides that a gold nanoparticle hinders a morphological change of a coronavirus protein, to block the entry of a coronavirus protein into a cell, resulting in inhibition of the cellular entry of the coronavirus.

The present invention also provides a pharmaceutical composition for treating or preventing COVID-19, comprising a gold nanoparticle.

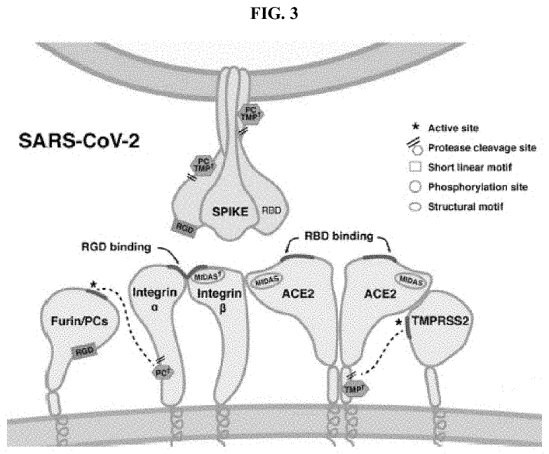

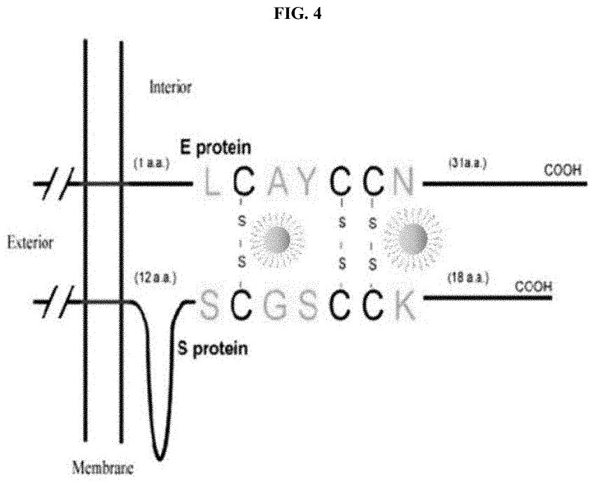

As shown in FIG. 3, the gold nanoparticle hinders the morphological change in a coronavirus protein to block the entry of the coronavirus protein into a cell. According to such blocking, COVID-19 may be treated or prevented. In further detail, as shown in FIG. 4, a negatively-charged colloidal gold solution may be inserted between a spike protein and an envelope protein. The insertion of the gold nanoparticle makes the morphological change of the spike protein difficult.

In the present invention, the gold nanoparticle may be provided in the form of a negatively-charged gold colloidal solution. When the gold colloidal solution is used through spraying into the nasal cavity, the viral entry into cells may be more effectively prevented, but there are no limitations on the administration method.

In the present invention, the gold nanoparticle has a diameter of 0.05 to 100 nm, preferably 0.1 to 50 nm, more preferably 0.1 to 35 nm, and most preferably 0.1 to 10 nm.

The “coronavirus-19”, which is a causative virus of a disease to be prevented or treated by the composition of the present invention, is a novel type of virus first identified in Wuhan, China in 2019, and the genomic and protein information associated with the coronavrius-19 may be freely collected from a research information database and used.

The term “prevention” used herein refers to all actions of suppressing COVID-19 or delaying the onset thereof by administration of the pharmaceutical composition according to the present invention.

The term “treatment” used herein refers to all actions involved in alleviating or beneficially changing symptoms of COVID-19 by administration of the pharmaceutical composition according to the present invention.

The present invention provides the simplest method of blocking the entry of a spike protein into a cell by hindering the morphological change of the protein using negatively-charged colloidal gold entering from an endotracheal tube or a nasal sprayer to the lungs. Here, small amounts of gold particles may migrate into the systemic circulatory system (10 to 40 nm).

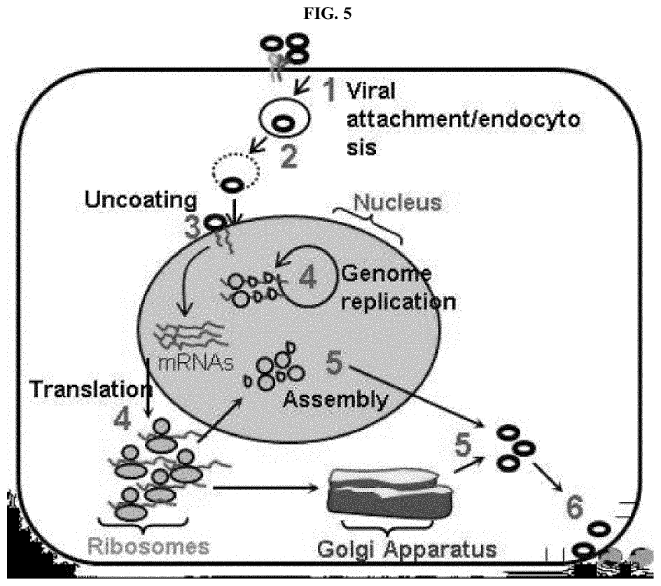

FIG. 5 shows that, when replicated coronavirus-19 is released from lung cells, a negatively-charged colloidal gold nanoparticle may easily bind to the positively-charged replicated virus, the virus may then be removed by phagocytosis, and thus the progression of infection may be prevented.

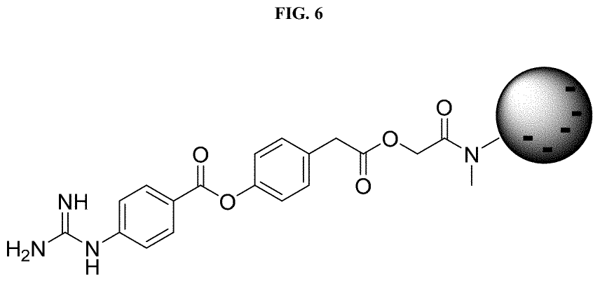

In addition, the gold nanoparticle of the present invention may be used in combination with an S1 proteolysis inhibitor. As shown in FIG. 6, camostat, which is the S1 proteolysis inhibitor, and the negatively-charged gold nanoparticles may be combined by ionic bonding, and therefore, the efficacy of the S1 proteolysis inhibitor may increase. That is, the progression of infection caused by coronavirus-19 may be inhibited by bonding between the gold nanoparticle acting as a protein form blocker and the S1 proteolysis inhibitor. The S1 proteolysis inhibitor may be, as an example, camostat mesylate, but the present invention is not limited thereto.

The gold colloidal solution provided in the present invention refers to a colloidal solution or suspension of submicrometer-sized gold nanoparticles, present in a fluid, generally, water.

The pharmaceutical composition according to the present invention may include a pharmaceutically acceptable carrier in addition to the active ingredient. Here, the pharmaceutically acceptable carrier is commonly used in preparation, and includes lactose, dextrose, sucrose, sorbitol, mannitol, starch, acacia gum, calcium phosphate, alginate, gelatin, calcium silicate, microcrystalline cellulose, polyvinyl pyrrolidone, cellulose, water, syrup, methyl cellulose, methylhydroxybenzoate, propyl hydroxybenzoate, talc, magnesium stearate and mineral oil, but the present invention is not limited thereto. In addition, the pharmaceutical composition according to the present invention may further include a lubricant, a wetting agent, a sweetening agent, a flavor, an emulsifier, a suspending agent or a preservative, in addition to the above-mentioned components.

The pharmaceutical composition of the present invention may be orally or parenterally (e.g., intravenously, subcutaneously, intraperitoneally or locally) administered by a desired method, and a dosage may vary according to a patient's condition and weight, the severity of a disease, a dosage form, an administration route and duration, but may be appropriately selected by those of ordinary skill in the art.

The pharmaceutical composition of the present invention may be administered at a pharmaceutically effective amount. The “pharmaceutically effective amount” used herein refers to an amount sufficient for treating a disease at a reasonable benefit/risk ratio applicable for medical treatment, and an effective dosage may be determined by parameters including a type of a patient's disease, severity, drug activity, sensitivity to a drug, administration time, an administration route and an excretion rate, the duration of treatment and drugs simultaneously used, and other parameters well known in the medical field. The pharmaceutical composition of the present invention may be administered separately or in combination with other therapeutic agents, and may be sequentially or simultaneously administered with a conventional therapeutic agent, or administered in a single or multiple dose(s). In consideration of all of the above-mentioned parameters, it is important to achieve the maximum effect with the minimum dose without a side effect, and such a dose may be easily determined by one of ordinary skill in the art.

Specifically, the effective amount of the pharmaceutical composition of the present invention may be dependent on a patient's age, sex, condition and body weight, an absorption rate of the active ingredient in the body, an inactivation rate, an excretion rate, a type of disease, or a drug used in combination, and may be generally administered at 10 to 1000 mg/kg, preferably 50-750 mg/kg, most preferably 100 to 500 mg/kg of body weight daily or every other day, or divided into one or three doses. However, the effective amount may vary depending on an administration route, the severity of obesity, sex, body weight or age, and therefore, the scope of the present invention is not limited by the dose in any way.

In addition, the present invention provides a method of treating or preventing coronavirus infection or COVID-19, which includes administering a composition including a gold nanoparticle to a subject. The term “subject” used herein refers to a target in need of treatment, and more specifically, a mammal such as a human or a non-human primate, a mouse, a rat, a dog, a cat, a horse, or a cow.

Furthermore, the present invention may provide a use of a composition including a gold nanoparticle to treat or prevent coronavirus infection or COVID-19.

Hereinafter, to help in understanding the present invention, an exemplary example will be suggested. However, the following example is merely provided to more easily understand the present invention, and not to limit the present invention.

### Examples

To confirm the effect of the composition of the present invention, a composition including 2-nm gold particles was prepared. The effect of the composition including the gold nanoparticle was studied by intratracheal administration to an adult female mouse. As a result, 1 hour after the single administration, the administered gold nanoparticles were found in lung macrophages.

From the above result, it can be confirmed that the inert gold nanoparticles administered into the trachea are involved in phagocytosis mediated by lung macrophages.

That is, a negatively-charged colloidal gold solution may approach coronavirus which has positively-charged porosity and hinders the morphological change of S1 and S2 domains by covalent binding to a cysteine residue of a spike protein, demonstrating that coronavirus-19 particles that are not able to be fused to lung cells through viral attachment to a host receptor are likely to be phagocytosed by lung macrophages.

It should be understood by those of ordinary skill in the art that the above description of the present invention is exemplary, and the exemplary embodiments disclosed herein can be easily modified into other specific forms without departing from the technical spirit or essential features of the present invention.

Therefore, the exemplary embodiments described above should be interpreted as illustrative and not limited in any aspect.

### INDUSTRIAL APPLICABILITY

The composition of the present invention is a pharmaceutical composition for treating or preventing coronavirus disease-19 (COVID-19), including a gold nanoparticle, and may block the entry of a coronavirus protein into a cell by hindering the morphological change of the spike protein of the coronavirus when the gold nanoparticles are administered. Therefore, the composition may be effectively used in the field of treating a novel infectious disease, such as COVID-19.

1. A method of treating or preventing coronavirus disease-19 (COVID-19) comprising:
administering a composition comprising a gold nanoparticle as an active ingredient, which comprises a gold nanoparticle as an active ingredient to a subject.

2. The method of claim 1, wherein the gold nanoparticle hinders the morphological change of a coronavirus protein to block the entry of the coronavirus protein into cells.

3. The method of claim 1, wherein the gold nanoparticle is included in the form of a negatively-charged gold colloidal solution.

4. The method of claim 1, wherein the composition further comprises an S1 proteolysis inhibitor.

5. The method of claim 1, wherein the composition is in a form of a nasal spray.

6. The method of claim 1, wherein the gold nanoparticle has a diameter of 0.1 to 100 nm.
